(19) United States
(12) Patent Application Publication
(10) Pub. No.: US 2011/0129872 A1
(43) Pub. Date: Jun. 2, 2011

(54) METHOD FOR A PRODUCTION OF A RECOMBINANT PROTEIN USING YEAST CO-EXPRESSION SYSTEM

(75) Inventors: Hyung Kwon LIM, Yongin-si (KR); Jin-Ho SEO, Seoul (KR); Yong-Cheol PARK, Seoul (KR)

(73) Assignee: MOGAM BIOTECHNOLOGY RESEARCH INSTITUTE, Yongin-si (KR)

(21) Appl. No.: 12/628,767

(22) Filed: Dec. 1, 2009

(51) Int. Cl. C12P 21/06

(52) U.S. Cl. 435/69.1

(57) ABSTRACT

The present invention relates to a method for mass production of a recombinant protein comprising the step of culturing a yeast transformed with: a recombinant gene construct comprising a yeast promoter, a gene coding a signal sequence and a gene coding a target protein; and also with one or more genes coding folding accessory protein selected from the group consisting of PDI1 (protein disulfide isomerase 1), SEC23 (secretory 23), TRX2 (thioredoxin 2) AHA1 (activator of heat shock protein 90 ATPase), and SCJ1 (S. cerevisiae DnaJ) followed by culturing the transformed yeast.

### FIELD OF THE INVENTION

The present invention relates to a method for a production of a recombinant protein using yeast co-expression system.

### BACKGROUND OF THE INVENTION

The production of a heterologous target protein in eukaryotic host cells is advantageous in that it allows the target proteins to fold and secret through their secretory machinery (Sarramegna et al., “Heterologous expression of G-protein-coupled receptors: comparison of expression systems from the standpoint of large-scale production and purification,” Cell Mol. Life Sci. 60:1529-1546, 2003). A number of host organisms such as Pichia pastoris, Kluyveromyces lactis, Hansenula polymorpha, and Saccharomyces cerevisiae have been used. Among them, S. cerevisiae is one of the most popular workhorses already used in food and biomedical industries. S. cerevisiae, GRAS (generally recognized as safe) microorganism, can be easily manipulated genetically. Genomic databases, tools and methods for genetic manipulations for yeast S. cerevisiae have been well established. However, this yeast inherently exhibits lower protein expression levels than prokaryotic host cells. To solve this problem, methods for chromosomal integration of multiple copies of a target gene have been developed employing transposable elements (Lee and DaSilva, “Improved efficiency and stability of multiple cloned gene insertions at the delta sequences of Saccharomyces cerevisiae,” Appl. Microbiol. Biotechnol., 48(3):339-3451997) and they enabled the high level of expression of the E. coli lacZ gene in S. cerevisiae.

It has been frequently observed that a recombinant S. cerevisiae strain retains most of the produced proteins in the endoplasmic reticulum (ER), where the yeast secreted a increased amount of the target protein (Cha et al., “Coexpression of protein disulfide isomerase enhances production of kringle fragment of human apoliprotein in recombinant Saccharomyces cerevisiae,” J. Microbiol. Biotechnol., 16(2):308-311, 2006; Kim et al., “Coexpression of BiP increased antithrombotic hirudin production in recombinant Saccharomyces cerevisiae,” J. Biotechnol., 101(1):81-87, 2003). Generally, overproduction of heterologous proteins is accompanied by the accumulation of improperly folded proteins, resulting in an ER-overload (Mattanovich et al. 2004).

Specifically, a series of secretion steps, so called, “the secretion pathway” is not a simple targeting of the materials to the exterior. In fact, the secretion of a target heterologous protein occurs in several compartmentalized organelles in eukaryotic organisms (FIG. 1). Protein secretion is directed by an amino-terminal signal sequence which mediates co-translational translocation of the target protein into the ER. The signal peptide is removed by a signal peptidase. In the lumen of the ER, asparagines-linked glycosyl structures may be added. The signal for the addition of the N-linked sugars is identical to those for yeast and mammalian glycoproteins (Asn-X-Ser/Thr). O-linked oligosaccharides may also be added. Proteins are then transported in vesicles to the Golgi where modifications to these glycosyl structures take place. Such modifications differ from those made by higher eukaryotic cells, and therefore, glycosylation is regarded as a major problem to be solved for the secretion of a therapeutic glycoprotein from yeast. From the Golgi, proteins are packaged into secretory vesicles and are delivered to the cell surface.

It is believed that, in an ER, a default pathway directs a protein to the plasma membrane unless it contains specific signals to cause retention in the ER or Golgi, or to target it to the vacuole. Therefore, it might be considered that if a foreign protein could be targeted to the lumen of the ER, it would be successfully secreted to the membrane. However, there are a number of stages in the secretory process where some problems may be encountered (Romanos et al., “Foreign gene expression in yeast: a review,” Yeast, 8:423-488 1992). The yeast proteins which assist in folding and disulfide bond formation differ from their counterparts in higher eukaryotes and this may affect folding of a recombinant protein. Malfolding can result in retention of the recombinant protein in the ER and their degradation (Biemans et al., “The large surface protein of hepatitis B virus in retained in the yeast endoplasmic reticulum and provokes unique enlargement,” DNA and cell biology, 10(33):191-200, 1991; Kim et al., “Coexpression of BiP increased antithrombotic hirudin production in recombinant Saccharomyces cerevisiae,” J. Biotechnol., 101(1):81-87, 2003). A recombinant protein can be secreted only when its folding and modification are precisely accomplished.

To date, various studies on secretion factors have been conducted to enhance the secretion of a recombinant protein in S. cerevisiae. The studies can be broadly divided into two categories: studies on trans-chaperones for promoting the folding of proteins newly synthesized in reticula and studies on cis-protein fusion factors for preventing overexpressed proteins from agglomerating in reticula.

In order to promote the secretion and growth of proteins in yeasts foldase and chaperone are required. In normal culture, such mediator proteins are not deficient. But in recombinant strains, as larger recombinant proteins are expressed, the mediator proteins can be depleted. For this reason, a method of overexpressing the folding-mediated foldase and chaperone has been developed to increase the secretion of recombinant proteins.

However, there still exists a strong need to develop an improved expression system employing such secretion factors in order to improve productivity of the recombinant protein. The experiment and analysis of a co-expression system for yeasts using specific secretion factors has not been reported yet.

The present inventors have endeavored to develop a method for mass production of a recombinant protein and found that a recombinant protein can be effectively produced by culturing a yeast transformed with a recombinant gene construct comprising a yeast promoter, a gene coding a signal sequence and a gene coding a target protein, and one or more genes coding folding accessory proteins selected from the group consisting of PDI1 (protein disulfide isomerase 1), SEC23 (secretory 23), TRX2 (thioredoxin 2), AHA1 (activator of heat shock protein 90 ATPase), and SCJ1 (S. cerevisiae DnaJ).

### SUMMARY OF THE INVENTION

Accordingly, it is an object of the present invention to provide a method for mass production of a recombinant protein employing a co-expression system of yeast.

In accordance with one aspect of the present invention, there is provided a method for producing a target protein comprising the step of culturing a yeast transformed with: a recombinant gene construct comprising a yeast promoter, a gene coding a signal sequence, and a gene coding a target protein; and also with one or more genes coding folding accessory proteins selected from the group consisting of PDI1 (protein disulfide isomerase 1), SEC23 (secretory 23), TRX2 (thioredoxin 2), AHA1 (activator of heat shock protein 90 ATPase), and SCJ1 (S. cerevisiae DnaJ), followed by the step of culturing the transformed yeast.

### BRIEF DESCRIPTION OF THE DRAWINGS

The above and other objects and features of the present invention will become apparent from the following description of the invention, when taken in conjunction with the accompanying drawings described below.

FIG. 1 shows the secretory pathway of foreign proteins in S. cerevisiae;

FIG. 2 is the graph for culture profiles of S. cerevisiae strains used in DNA microarray;

FIG. 3 shows the graphs for DNA microarray analysis data;

FIG. 4 shows genetic map of MδCL-HBs;

FIG. 5 shows plasmid maps with the genes coding folding accessory proteins;

FIG. 6 shows the immunoblots of HBsAg co-expressed with protein folding accessories (1: Control, HBsAg expression without co-expression, 2: HBsAg expression with PDI1 co-expression, 3: HBsAg expression with SEC23 co-expression, 4: HBsAg expression with TRX2 co-expression, 5: HBsAg expression with AHA1 co-expression, 6: HBsAg expression with SCJ1 co-expression);

FIG. 7 shows plasmid maps for co-expression of four kinds of combination, HBsAg expression with PDI1 and TRX2, HBsAg expression with PDI1 and SEC23, HBsAg expression with PDI1, TRX2, and SEC23, HBsAg expression with TRX2 and SEC23;

FIG. 8 shows the immunoblots of HBsAg co-expressed with protein folding accessories (STD: 5 mg/L HBsAg standard, P: HBsAg expression with PDI1 co-expression, S: HBsAg expression with SEC23 co-expression, T: HBsAg expression with TRX2 co-expression, PT: HBsAg expression with PDI1 and TRX2 co-expression, PS: HBsAg expression with PDI1 and SEC23 co-expression, ST: HBsAg expression with SEC23 and TRX2 co-expression, PST: HBsAg expression with PDI1, SEC23 and TRX2 co-expression);

FIG. 9 shows plasmid maps for KEX2 expression;

FIG. 10 shows the immunoblots of HBsAg co-expressed with KEX2 (1: Control, HBsAg expression with PDI co-expression, 2: HBsAg expression with PDI1 and inducible KEX2 co-expression, 3: HBsAg expression with PDI1 and constitutive KEX2 co-expression);

FIG. 11 shows the immunoblots of HBsAg co-expressed with KEX2;

FIG. 12 shows batch fermentation profiles. Each symbol represents cell growth (◯), residual galactose (Δ), residual ethanol (∇), residual glucose (□), total HBsAg concentration (♦);

FIG. 13 shows fed-batch fermentation profiles of recombinant S. cerevisiae 2805/δCL-HBs_PDI1;

FIG. 14 shows fed-batch fermentation profiles of PT and PTK strains. Each symbol represents cell growth (◯), residual galactose (Δ), residual ethanol (∇), residual glucose (□), total HBsAg content (♦);

FIG. 15 shows fed-batch fermentation profiles of PS and PSK strains. Each symbol represents cell growth (◯), residual galactose (Δ), residual ethanol (∇), glucose (□), total HBsAg content (♦);

FIG. 16 shows the immunoblots of HBsAg during the fed-batch fermentation (STD: 5 mg/L HBsAg standard, Control: HBsAg expression (Batch fermentation sample, 60 hr), PT: HBsAg expression with PDI1 and TRX2 co-expression, PTK: HBsAg expression with PDI1, TRX2 and KEX2 co-expression, PS: HBsAg expression with PDI1 and SEC23 co-expression, PSK: HBsAg expression with PDI1, SEC23 and KEX2 co-expression);

FIG. 17 shows comparison of quantitative western blot and ELISA results; and

FIG. 18 shows the results of transmission electron micrographs (A: S. cerevisiae 2805/MδCL-HBs_PDI, B: S. cerevisiae 2805/MδCL-HBs_PSK)

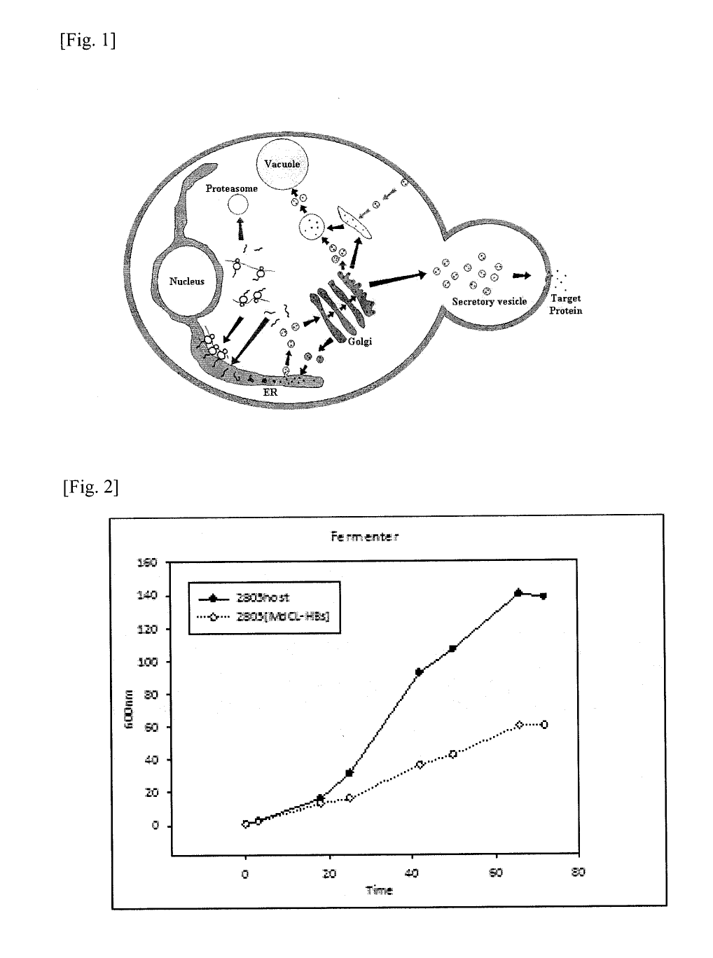

### DETAILED DESCRIPTION OF THE INVENTION

In the present invention, there is provided a method for producing a target protein comprising the step of culturing a yeast transformed with: a recombinant gene construct comprising a yeast promoter, a gene coding a signal sequence, and a gene coding a target protein; and one or more genes coding folding accessory protein selected from the group consisting of PDI1 (protein disulfide isomerase 1), SEC23 (secretory 23), TRX2 (thioredoxin 2), AHA1 (activator of heat shock protein 90 ATPase), and SCJ1 (S. cerevisiae DnaJ), followed by the step of culturing the transformed yeast.

The term “promoter” as used herein, refers to a DNA fragment determining a transcription starting point of a gene under control of said promoter and starting frequency. The yeast promoter of the subject invention is preferably galactose-induced promoter such as GAL 1, GAL 7 and GAL 10 promoter. The preferred galactose-induced promoter is GAL 1 promoter. The GAL 1 promoter is a strong tightly-regulated promoter of S. cerevisiae and involved in galactose metabolism. Since the GAL1 promoter is strongly repressed by glucose, maximal induction can be achieved by depletion of glucose.

The recombinant gene construct also comprises a gene coding a signal sequence. The “signal sequence” as used herein, is understood to mean a segment which directs the secretion of the biologically active molecule. The signal sequence used in the present invention may be a polynucleotide which encodes an amino acid sequence initiating transport of a protein across the membrane of the endoplasmic reticulum (ER). The non-limiting examples of the signal sequence are MFα (mating factor α signal sequence), K1 killer toxin signal, invertase secretion signal peptide, killer toxin of Kluyveromyces lactis signal sequence, killer toxin of Pichia acaciae signal sequence, killer toxin of Hanseniaspora uvarum signal sequence, and killer toxin of Pichia (Hansenula) anomala signal sequence. The preferred signal sequence of the subject invention is MFα (mating factor α signal sequence). Preferably, for a correct folding and translocation of a target protein, MFα signal peptide is introduced. MF α is the pre-pro region from α-factor, and encodes a protein having 165 amino acids, pre-pro-α-factor, which comprises a signal sequence of 19 amino acids (the pre region) and a pro region, followed by four tandem repeats of the mature 13 amino acid α-factor sequence (Romanos et al. 1992).

The recombinant gene construct also contains a gene coding a target protein, preferably a gene coding a hepatitis B type surface antigen, which can be composed of only S region (Small), or S with pre-S2 (medium), or S with pre-S1 and pre-S2 (large).

In the recombinant gene construct of the present invention, the above genes coding MFα and a target protein are operably linked to the GAL 1 promoter.

According to the present method, the yeast is also transformed with one or more genes coding folding accessory proteins selected from the group consisting of PDI1 (protein disulfide isomerase 1), SEC23 (secretory 23), TRX2 (thioredoxin), AHA1 (activator of heat shock protein 90 ATPase), and SCJ1 (S. cerevisiae DnaJ) which are screened by DNA microarray using RNA sample of cells harvested during the expression of HBsAg. All of them are known as molecular chaperones and foldases. Preferably, the gene coding folding accessory proteins is PDI1, SEC 23 or TRX2. The gene coding folding accessory proteins for the present invention may be used alone, or in combination of two or more. The preferred combination of said genes is PDI1 and SEC23, PDI1 and TRX2, or PDI1, SEC23 and TRX1. Theses genes coding folding accessory proteins facilitate disulfide bond formation or induce protein transport in the secretion pathway of the yeast, thereby improving the secretion of the recombinant target protein.

To increase the expression level of the recombinant protein, a gene coding KEX 2 (kexin 2) protease may be further introduced in the yeast. KEX2 protease is a Ca²⁺ dependent serine protease involved in proprotein processing (Fuller R S et al., 1989). KEX2 protease cleaves target peptides at paired dibasic sites and is required to produce the mature forms of secreted proteins such as alpha-factor (Julius D et al., 1984).

In general, the construction of genes disclosed herein is achieved by known method utilized in genetic engineering technology.

In the method according to the present invention, the yeast may be transformed with either a single vector comprising both of the recombinant gene construct and one or more genes coding folding accessory proteins, or separate vectors comprising the recombinant gene construct and one or more genes coding folding accessory proteins, respectively.

As used herein, the term “vector” is understood to mean any nucleic acid molecule including a nucleotide sequence competent to be incorporated into a host cell and an integrated into the host cell genome, or to replicate autonomously as an episome. Such vectors include linear nucleic acids, plasmids, phagemids, cosmids, RNA vectors, viral vectors and the like. Suitable expression vector may comprise expression regulatory factor such as promoter, start codon, stop codon, polyadenylation signal, enhancer and selection markers.

The transformation of the recombinant gene construct and one or more genes coding folding accessory proteins into the yeast may be conducted by known method in the art, which may be selected suitably depending on host cells. These methods include, but not limited to, electroporation, protoplast fusion method, calcium phosphate (CaPO₄) precipitation and calcium chloride (CaCl₂) precipitation, agitation with silicon carbide fiber, and PEG-, dextran sulfate- and lipofectamine-mediated transformation. The yeast to be transformed is preferably Saccharomyces cerevisiae, more preferably S. cerevisiae 2805. In one aspect of the present invention, the recombinant gene construct and one or more genes coding folding accessory protein are individually introduced into S. cerevisiae 2085, thus generating the transformants described in Table 1.

Meanwhile, for culturing the above transformed yeast for co-expression, any known methods for culturing yeasts can be used. Examples of known culturing methods are batch culture, continuous culture and fed-batch culture. In order to maximize the expression of the recombinant protein, the fed-batch culture method is preferred. Culture conditions suitable for selected yeast strains may be easily adjusted by those skilled in the art. Typically, a medium used in the culturing should contain all nutrients essential for the growth and survival of cells. The medium should contain a variety of carbon sources, nitrogen sources and trace elements. Examples of available carbon sources include glucose, galactose, sucrose, lactose, fructose, maltose, starch, carbohydrates such as cellulose, fats such as soybean oil, sunflower oil, castor oil and coconut oil, and alcohols such as glycerol and ethanol. These carbon sources may be used alone or in combination of two or more. Glucose and galactose may be preferably used as the carbon source for culturing the present transformed yeast. The concentration of the carbon source may be controlled. Preferably, the concentration of glucose is maintained in the range of less than 1 g/L, and the concentration of galactose is maintained in the range of 10 to 50 g/L during induction.

As can be seen from the above, the method for producing a recombinant protein according to the present invention employing the above recombinant construct and the gene coding the folding accessory protein, can reduce the accumulation of improperly folded proteins accompanied by the overproduction of the recombinant protein, resulting in an ER-overload, and enhance productivity of the recombinant protein in the yeast expression system.

The present invention will be described in further detail with reference to the following Examples. However, it should be understood that the present invention is not restricted by the specific Examples.

### EXAMPLE

### <Materials and Methods>

### 1. Strains and Plasmids

E. coli Top10 [F-mcrA Δ(mrr-hsdRMS-mcrBC) φ80lacZΔM15 ΔlacX74 recA1 araD139 Δ(ara-leu)7697 galU galK rpsL (StrR) endA1 nupG] was used for genetic manipulation. S. cerevisiae 2805 [MATα pep4::HIS3 prb-Δ1.6 his3 ura3-52 can1] is used as a yeast host for the HBsAg gene expression. A recombinant S. cerevisiae 2805/MδCL-HBs strain was constructed to contain the HBsAg expression cassette comprised of the GAL1 promoter, α-factor signal sequence, HBsAg structural gene and CYC1 terminator. All recombinant S. cerevisiae strains used in the present invention are listed in Table 1.

Specifically, MδLK8 plasmid was prepared according to Korean Patent Nos. 595864 and 672745 and was treated with XhoI for truncating LK8 gene. After gel purification, remaining large fragment (about 5.9 kb) was purified and ligated to gene coding HBsAg, to prepare expression vector MδCL-HBs. The MδCL-HBs expression vector was integrated into S. cerevisiae 2805 or S. cerevisiae BJ3501 (Korean Patent No. 672745) and colonies were selected with 1 mg/ml of G418. For obtaining high copy number transformant, 112 clones were screened by increasing the concentration of G418 such as 1 mg/ml, 5 mg/ml and 10 mg/ml, and 75 clones among them were tested for the secretion level of HBsAg in 5 ml of medium (4% yeast extract, 0.5% casamino acid, 0.05% uracil, 0.05% histidine, 0.5% glucose, 2% galactose). Then, the strain exhibiting the most excellent expression was selected by enzyme-linked immunosorbent assay using culture supernatant, and named as S. cerevisiae 2805/MδCL-HBs strain.

Plasmid p426Gal1, pAUR123, pMPdI1 were used as mother vectors to carry each gene. Plasmid TRX2-p426Gal1, SEC23-p426Gal1, SCJ1-p426Gal1, AHA1-p426Gal1, and pMPDI1 were constructed for the expression of TRX2, SEC23, SCJ1, AHA1, and PDI1, respectively. PCR was carried out using genomic DNA isolated from S. cerevisiae as a template and oligonucleotide described in Table 2 as a primer. Plasmid pMPDI1TRX2, pMPDISEC23, and TRX2-pAUR123 were constructed for combinatorial expression of folding accessories. Plasmid KEX2-p426Gal1, KEX2-pAUR123, and pMPDI1KEX2 were constructed for KEX2 co-expression with HBsAg. These vectors are prepared by cloning the genes coding folding accessory protein, i.e., PDI1, SEC23, AHA1, SCJ1 and TRX2 into p426GAL1 (ATCC87833, USA), pMPD1 (Korean Patent No. 672745), and pAUR123 (Takara Bio).

TABLE 1
Recombinant S. cerevisiae strains used herein

| Strain | Genotype | Reference |
|---|---|---|
| S. cerevisiae 2805 | MATαpep4::HIS3 prb-Δ1.6 his3 ura3-52 can1 | Sohn, J. H. et al. Proc. |
| | | Biochem, 30: 653-660, |
| | | 1995; Kim, T. H. et al., |
| | | Biotechnol. Lett., 24: 279- |
| | | 286, 2002 |
| 2805/MδCL-HBs | 2805 Ty-δ::P_{GAL1}-MFα-HBsAg-T_{cyc1}-neo^{r} | present invention |
| 2805/MδCL-HBs_PDI1 | 2805/MδCL-HBs, pMPDI1 | present invention |
| 2805/MδCL-HBs_TRX2 | 2805/MδCL-HBs, TRX2-p426Gal1 | present invention |
| 2805/MδCL-HBs_SEC23 | 2805/MδCL-HBs, SEC23-p426Gal1 | present invention |
| 2805/MδCL-HBs_AHA1 | 2805/MδCL-HBs, AHA1-p426Gal1 | present invention |
| 2805/MδCL-HBs_SCJ1 | 2805/MδCL-HBs, SCJ1-p426Gal1 | present invention |
| 2805/MδCL-HBs_PT | 2805/MδCL-HBs, pMPDI1TRX2 | present invention |
| 2805/MδCL-HBs_PS | 2805/MδCL-HBs, pMPDI1SEC23 | present invention |
| 2805/MδCL-HBs_ST | 2805/MδCL-HBs, SEC23-p426Gal1, TRX2-pAUR123 | present invention |
| 2805/MδCL-HBs_PST | 2805/MδCL-HBs, pMPDI1SEC23, TRX2-pAUR123 | present invention |
| 2805/MδCL-HBs_pMPK | 2805/MδCL-HBs, pMPDI1KEX2 | present invention |
| 2805/MδCL-HBs_PK | 2805/MδCL-HBs, pMPDI1, KEX2-pAUR123 | present invention |
| 2805/MδCL-HBs_TK | 2805/MδCL-HBs, TRX2-p426Gal1, KEX2-pAUR123 | present invention |
| 2805/MδCL-HBs_SK | 2805/MδCL-HBs, SEC23-p426Gal1, KEX2-pAUR123 | present invention |
| 2805/MδCL-HBs_PTK | 2805/MδCL-HBs, pMPDI1TRX2, KEX2-pAUR123 | present invention |
| 2805/MδCL-HBs_PSK | 2805/MδCL-HBs, pMPDI1SEC23, KEX2-pAUR123 | present invention |

TABLE 2
Sequences of oligonucleotides used herein

| Name | Sequence | Relevant work | SEQ ID No. |
|---|---|---|---|
| F_Aha1_BamH I | CGCGGATCCATGGTCGTGAATAACCCAAAT | AHA1-p426Gal1 | 1 |
| R_Aha1_Hind III | CCCAAGCTTTTATAATACGGCACCAAAGCC | AHA1-p426Gal1 | 2 |
| F_Scj1_BamH I | CGCGGATCCATGATTCCAAAATTATATATACATT | SCJ1-p426Gal1 | 3 |
| R_Scj1_Hind III | CCCAAGCTTCTACAACTCATCTTTGAGCAT | SCJ1-p426Gal1 | 4 |
| F_Sec23_BamH I | CGCGGATCCATGGACTTCGAGACTAATGA | SEC23-p426Gal1, pMPDISEC23 | 5 |
| R_Sec23_Hind III | CCCAAGCTTCTATGCCTGACCAGAGAC | SEC23-p426Gal1 | 6 |
| R_Sec23_Xho I | CCGCTCGAGCGGCTATGCCTGACCAGAGAC | pMPDISEC23 | 7 |
| F_Trx2_BamH I | CGCGGATCCATGGTCACTCAATTAAAATCC | TRX2-p426Gal1, pMPDITRX2 | 8 |
| R_Trx2_Hind III | CCCAAGCTTCTATACGTTGGAAGCAATAG | TRX2-p426Gal1 | 9 |
| R_Trx2_Xho I | CCGCTCGAGCGGCTATACGTTGGAAGCAATAG | pMPDITRX2, TRX2-pAUR123 | 10 |
| F_Trx2_Kpn I | GGGGTACCCCATGGTCACTCAATTAAAATCC | TRX2-pAUR123 | 11 |
| F_Kex2_BamH I | CGGGATCCCGATGAAAGTGAGGAAATATATTAC | pMPDIKEX2 | 12 |
| R_Kex2_ Xho I | CCGCTCGAGCGGTCACGATCGTCCGGAAGAT | pMPDIKEX2 | 13 |
| F_Kex2_Xho I | CCGCTCGAGCGGATGAAAGTGAGGAAATATATTAC | KEX2-p426Gal1, KEX2-pAUR123 | 14 |
| R_Kex2_Xho I | CCGCTCGAGCGGTCACGATCGTCCGGAAGAT | KEX2-p426Gal1 | 15 |
| R_Kex2_Xba I | GCTCTAGAGCTCACGATCGTCCGGAAGAT | KEX2-pAUR123 | 16 |

### 2. Media and Culture Conditions

LB medium (1% NaCl, 1% tryptone, and 0.5% yeast extract) was used for E. coli cultivation. YPD solid medium (1% yeast extract, 2% peptone, 2% glucose and 2% agar) supplemented with filter-sterilized G418 sulfate (Q-BIO gene Inc., CA, USA) was used for the maintenance of yeast transformants harboring the G418 resistant gene. YNB solid medium without uracil (0.67% yeast nitrogen base without amino acid, 0.192% supplement of amino acids without uracil, 2% glucose, and 2% agar) (Burke et al. 2000) was used for the selection of the transformants possessing the URA3 gene. For the transformants with the Aureobasidin A resistant genes, YNB solid medium lacked uracil was supplemented with o.6 g/L AbA (AbA, TaKaRa Inc., Tokyo, Japan).

A bench-top fermentor (Bioengineering AG, Wald, Switzerland) was used for batch and fed-batch cultivations. Batch and fed-batch fermentations were performed in one liter of YPDG medium (1% yeast extract, 2% peptone, 2% glucose and 3% galactose). An agitation speed of 500 rpm and an aeration of 1 vvm were maintained throughout the cultivation. Medium pH was adjusted at 5.5 with 2N HCl or 2N NaOH, and temperature was maintained at 30° C. For fed-batch fermentation, glucose (60%, w/v) was constantly fed into the reactor as the carbon source after depletion of the glucose initially added. And 250 g/L galactose was intermittently fed into the medium to maintain the galactose level over 1.0% (w/v).

### 3. DNA Manipulation and Transformation

### 3.1. Enzymes

Restriction enzymes and calf intestinal alkaline phosphatase (CIP) were purchased from New England Biolabs (Beverly, Mass., USA). T4 DNA ligase kit obtained from Takara (Tokyo, Japan) was used for ligation.

3.2. Transformation of E. coli

In order to sub-cloning of PDI1, SEC23, AHA1, SCJ1 and TRX2, transformation of E. coli was carried out as described by Sambrook et al. (1989). E. coli Top10 was cultured in 5 ml Luria-Bertani (LB) medium (0.5% yeast extract, 1% tryptone, and 1% NaCl) for 12 hr. 500 μl of the culture was transferred to fresh 50 ml LB medium, which was cultured until OD at 600 nm reached the 0.5. Cells harvested by centrifugation at 3000 rpm at 4° C. for 5 min were resuspended in 5 ml of 100 mM ice-cold CaCl₂ solution containing 15% (v/v) glycerol. The resuspension of 100 ml were aliquoted, mixed with DNA (TRX2-p426Gal1, SEC23-p426Gal1, SCJ1-p426Gal1, AHA1-p426Gal1, pMPDI1, pMPDI1TRX2, pMPDISEC23, TRX2-pAUR123, KEX2-p426Gal1, KEX2-pAUR123, and pMPDI1KEX2), and kept on ice for 30 min. After heat-shock at 42° C. for 45 sec, 1 ml of LB medium was added to the resuspension prior to incubation at 37° C. for 1 hr. An appropriate volume of the transformed cells was spread on LB agar plates with ampicillin.

### 3.3. Preparation of Plasmid DNA and Yeast Genomic DNA

Mini-scale preparation of plasmid DNA was carried out using the Axyprep™ Plasmid Extraction Kit (Axygen Scientific Inc., CA, USA) according to the manufacturer's instruction.

Preparation of the yeast genomic DNA was carried out using the Masterpure™ Yeast DNA Purification Kit (EPICENTRE, WI, USA) according to the manufacturer's instruction.

### 3.4. Isolation of DNA Fragments and DNA Sequencing

DNA was digested with restriction enzymes and separated on 0.1% (w/v) agarose gel. After full separation of the desired DNA band from the gel, the gel containing the DNA fragment was solubilized and further purified by using the Gel Extraction Kit (Takara, Tokyo, Japan).

DNA sequencing for TRX2-p426Gal1, SEC23-p426Gal1, AHA1-p426Gal1, and SCJ1-p426Gal1 were performed by National Instrumentation Center for Environmental Management (Seoul, Korea).

DNA sequencing for KEX2-p426Gal1, pMPDI1TRX2, pMPDI1SEC23, pMPDI1KEX2, KEX2-pAUR123, and KEX2-pAUR123 were performed by SolGent (Daejon, Korea).

### 3.5. Polymerase Chain Reaction (PCR)

Polymerase chain reaction (PCR) was performed with the Accupower™ PCR PreMix (Bioneer Co., Daejon, Korea) in GeneAmp PCR System 2400 (Applied Biosystems, CA, USA). PCR solution was composed of 10 pmol of forward and reverse primers, and 10 ng genomic DNA or plasmid DNA as a template. PCR amplification was performed as follows; 1 cycle of 95° C. for 5 min; 30 cycles of 94° C. for 30 sec, 58° C. for 1 min, 72° C. 1 min or 2 min; 1 cycle of 72° C. for 7 min. The amplified gene was confirmed by gel electrophoresis.

### 3.6. Yeast Transformation

Yeast transformation was carried out by the Alkali-Cation method with following modifications to increase transformation efficiency. Recombinant yeast strains transformed with plasmids harboring the URA3 genes or AbA resistant genes were cultivated and selected on SC plates lacked uracil or YPD plates containing AbA, respectively.

### 4. Analytical Methods

Optical density was measured with a spectrophotometer (Ultrospec 2000, Amersham Phamacia Biotech., USA) at 600 nm. Concentrations of glucose, galactose, and ethanol were measured by a high performance liquid chromatography (Agilent 1100LC, USA) equipped with a RI detector. Samples were separated by the Carbohydrate Analysis column (Phenomenex, CA, USA), and HPLC operation conditions were set according to the instruction manual of the column supplier.

### 5. Immunoblot Analysis

Enhanced chemiluminescence (ECL) western blot was carried out for the determination of HBsAg content. Samples for intracellular HBsAg and KEX2 were prepared by adjusting the cell density in 1× phosphate-buffered saline (PBS, pH7, 0.15% Na₂HPO₄, 0.04% KH₂PO₄, 0.61% NaCl w/v) (10 OD₆₀₀/ml). The cell pellets were suspended in 2× SDS sample buffer, and disrupted by vortexing with 0.5 mm glass beads (Biospec Products, OK, USA). After 12 min boiling, crude sample was centrifuged at 12,000 rpm in a microcentrifuge for 5 min at 4° C. The pellet was discarded, and supernatant was loaded on 12% Sodium dodecyl sulfate-polyacrylamide gel. Purified recombinant HBsAg (Green Cross MS co., Chungbuk, Korea) was also loaded in parallel as a standard. After electrophoresis, the proteins were transferred to 0.45 μm polyvinylidene difluoride (PVDF) membranes (PALL co., NY, USA). Membranes were blocked with 5% milk powder in 1× PBS and incubated with goat anti-HBsAg (6 mg/ml, Green Cross MS co.) in blocking solution. Rabbit anti-goat horseradish peroxidase conjugate (Santa cruz Biotech., CA, USA) in blocking solution was used as the secondary antibody. HBsAg on the membrane was visualized with the Supersignal® West Pico Chemiluminescent substrate kit (PIERCE, IL, USA) and a medical X-ray film (Kodak, NY, USA). The quantity of HBsAg protein in an extracted sample was determined by comparing the densitometric signal present in lanes with that of standard HBsAg. Multiple Western blots (2-5 repetitions) were done to ensure reproducibility of the data. The band density was evaluated by densitometric scanning using Total Lab software (BioSystematica, UK) and values given in pixel unit were used for calculation. The specific content of HBsAg was calculated by the division of HBsAg concentration by dry cell mass.

### 6. Enzyme-Linked Immunosorbent Assay (ELISA)

Intracellular HBsAg was quantified by ELISA using HBsAg-specific polyclonal antibodies. The amount of the antigen in the cell extract was calculated using a standard curve based on various concentrations of purified HBsAg.

Samples for intracellular HBsAg were prepared by adjusting the cell density in 1× PBS (20 OD₆₀₀/ml). After the cells were separated by centrifugation, the cell pellets were suspended in disruption buffer (50 mM Tris, 150 mM NaCl, 1M

Potassium thiocyanate, 10 mM EDTA, 0.1% Tween-80 v/v) containing protease inhibitor (Roche, Switzerland). The cell membrane was disrupted by vortexing with 0.5 mm glass beads for 10 min. After vortexing, the suspension was centrifuged at 12,000 rpm in a microcentrifuge for 5 min at 4° C. The pellet was discarded, and the supernatant was subjected to ELISA. Analysis for each sample was done in duplicate.

For the plate ELISA assay, samples and standard were diluted in carbonate/bicarbonate buffer solution (0.16% Na₂CO₃, 0.3% NaHCO₃, 0.02% NaN₃); 100 μl of this sample was added into each well of the microtiter plate for coating, which was incubated at 4° C. overnight. The coat solution was discarded and 200 μl blotto (5% milk powder in 1× PBS) was added into each well to block the portions of the wall in the well that had not bound antigen. The plate was incubated at 37° C. for 2 h. After 2 h, the blotto was discarded and the plate was washed 3 times with PBS-T (1× PBS with 0.05% tween-20). Blotto was diluted 1:10 in 1× PBS to make 0.5% blotto. Goat anti-HBsAg was diluted 1:2000 in 0.5% blotto; 100 μl of the primary antibody was added into each well and incubated at 37° C. After 2 h, the plate was washed five times, and 100 μl of rabbit anti-goat-HRP, which was diluted 1:2000 in 0.5% blotto, was added into each well. The plate was incubated at 37° C. for 1 h and then washed five times with PBS-T. For color development, 100 μl of o-phenylenediamine dihydrochloride (OPD) as a peroxidase substrate was added into each well. After 10-15 min, the color in the control wells turned yellow, and 100 μl of 1N H₂SO₄ was added to stop the reaction. Absorbance was measured at 492 nm using a VERSAmax microplate reader (Molecular Device, CA, USA). All data were obtained from at least three or more independent experiments and averaged.

### 7. In Vitro KEX2 Treatment

The KEX2 protease was obtained in a membrane extract from S. cerevisiae 2805 strain overproducing KEX2 from a multicopy plasmid KEX2-p426Gal1. Yeast was grown to mid-logarithmic phase and the cells were harvested by centrifugation and broken by glass bead lysis in 50 mM Hepes/KOH (pH7.6). Cell debris was removed by centrifugation at 8000 rpm for 15 min at 4° C., and membranes were pelleted by centrifugation at 12000 rpm for 90 min at 4° C. These membranes were resuspended in 50 mM Hepes/Tris (pH7.6) containing 1% Triton X-100 and stored at −70° C. (Ledgerwood E C et al. 1995). HBsAg samples were prepared by the method of immunoblot assay. Incubation of membrane extract containing KEX2 protease with HBsAg protein samples was done at 30° C. for 4 hours.

### 8. Transmission Electron Microscopy (TEM) Analysis

Ten OD₆₀₀ unit equivalents of cells were resuspended in modified Karnovsky's fixative (2% paraformaldehyde and 2% glutaraldehyde in 0.05M sodium cacodylate buffer, pH7.2) at 4° C. for 2 hr. Cells were then centrifuged, washed three times with the 0.05M sodium cacodylate buffer (pH 7.2) before being post-fixed in post-fixative (1% osmium tetroxide in 0.05M cacodylate buffer, pH7.2) at 4° C. for 2 hr. The pellet was washed two times briefly with distilled water and left in the 0.5% uranyl acetate at 4° C. overnight (En bloc staining). Cells were dehydrated in increased amounts of ethanol (30, 50, 70, 80, 90, and three times 100%) by incubation in a rotatory wheel for at least 10 min at room temperature at each step. These amalgamations were followed by others: 100% ethanol:LR white resin (1:2) for 2 hr, 100% ethanol:LR white resin (1:3) for 2 hr, LR white resin for 2 hr, LR white resin overnight, LR white resin for 2 hr. After centrifugation, the LR white resin was polymerized by heating the sample at 55° C. for 24 hr. About 65-80 nm sections were then cut using a Vibratome (Technical products international) and transferred into carbon-coated copper grids.

For the immunolabeling, sections on girds were first blocked overnight by floating on drops of PBS containing 1% BSA, followed by washing with drops of PBST (PBS containing 0.1% Tween-20). This was followed by incubation for 2 hr with goat anti-HBs antibody diluted 1:1000. All antibody dilutions were made with PBS containing 1% BSA. Then the grids were washed and bound antibodies were localized by incubating the sections for 1 hr on anti-goat IgG gold conjugate (10 nm, Sigma, MO, USA). Finally, grids were washed with drops of PBST and distilled water before drying. The observation in a JEM1010 TEM (JEOL, Japan) was performed by National Instrumentation Center for Environmental Management (Seoul, Korea).

### Example 1

### Gene Expression Profiling of Relevant Genes

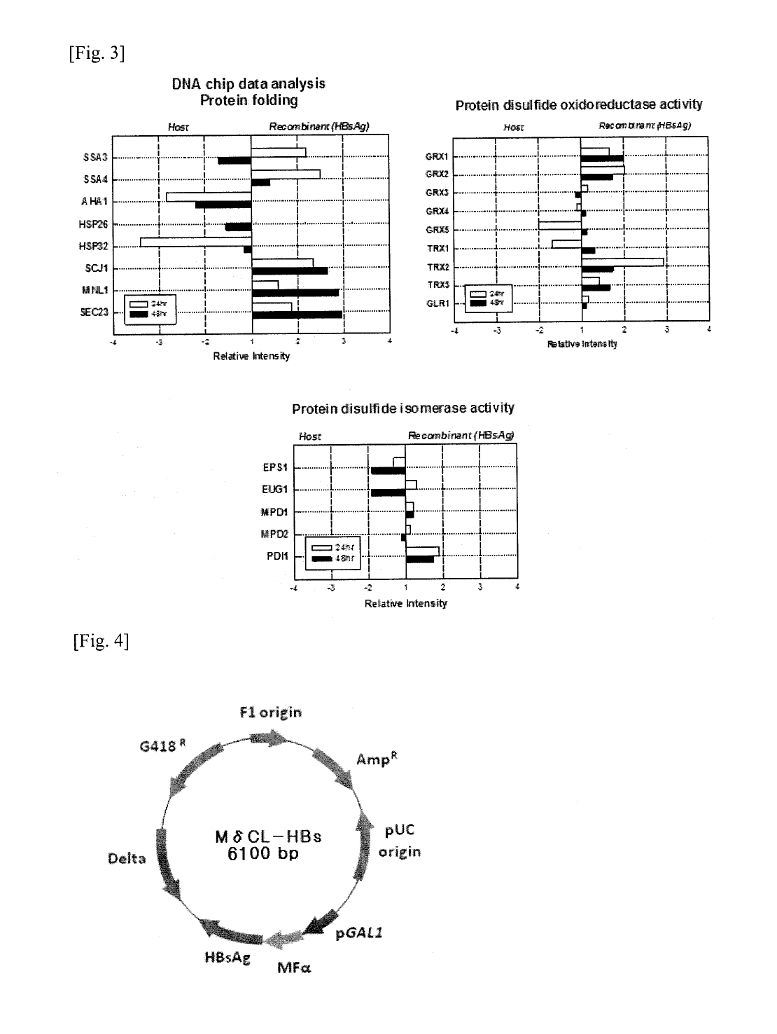

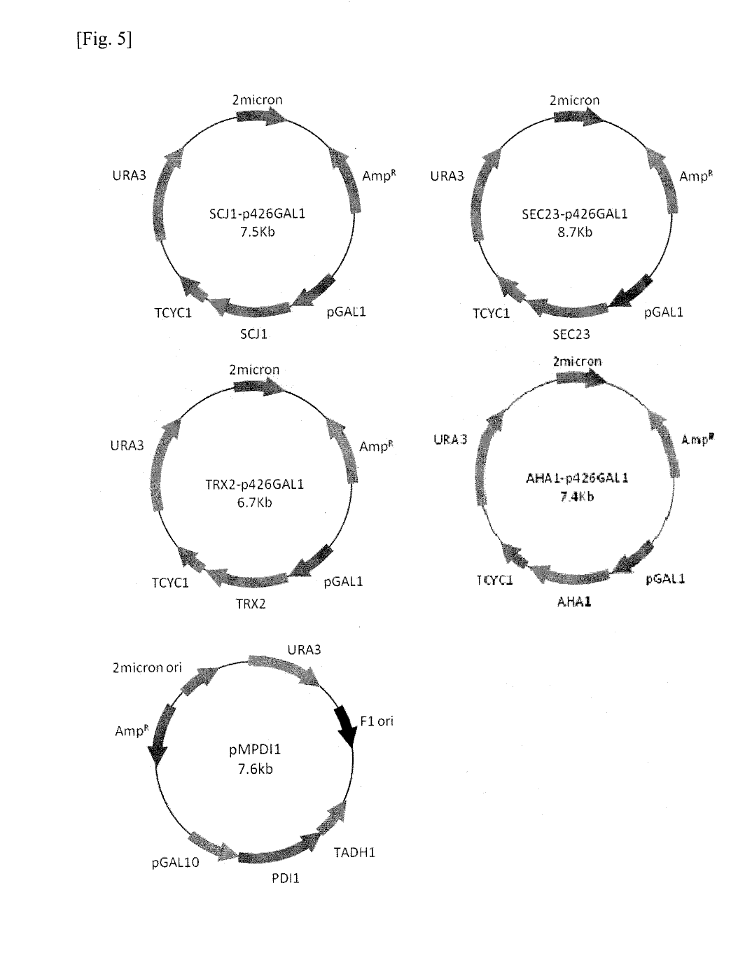

Effects of HBsAg expression on the S. cerevisiae genome were analyzed by monitoring quantitative gene expression patterns through DNA microarray. The strains used in this study are S. cerevisiae 2805 and S. cerevisiae 2805/MδCL-HBs. The cell was harvested at 24, 48 hr in the culture (FIG. 2) and the RNA was purified. The expression profiling was measured by using the custom-ordered microarray chip (CombiMatrix Corp, USA) being composed of 600 interested genes. Among the genes tested, only genes showing a change of level of RNA more than two-fold comparing the host S. cerevisiae 2805 were selected (FIG. 3) for the next co-expression experiments.

### Example 2

### Effects of Co-Expression of Protein Folding Accessories

To examine the effects of co-expresssion of folding accessory proteins on HBsAg production, S. cerevisiae 2805/MδCL-HBs was transformed with plasmids harboring the genes coding folding accessory proteins using the lithium cesium acetate method. As the HBsAg, PDI1, SEC23, TRX2, SCJ1, AHA1 encoding genes are located behind the GAL1 or GAL10 promoter, their expression are simultaneously induced by galactose (FIG. 4, 5). All transformants were selected on YNB solid medium without uracil and with 1 g/L G418 sulfate.

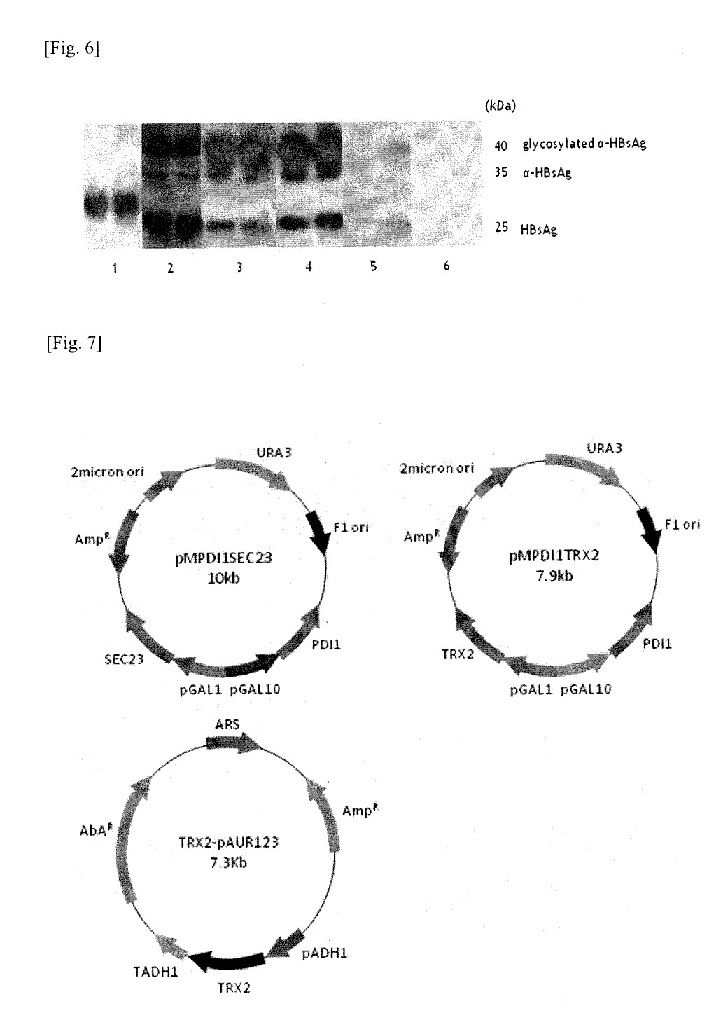

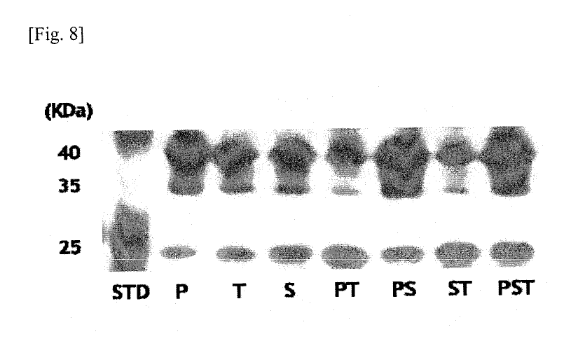

Immunoblot assay (FIG. 6) was performed to assess the effect of chaperone co-expression on the production of HBsAg. As a result, PDI1, SEC23, TRX2 co-expression seemed to make a profound effect on HBsAg production. Co-expression of the three folding proteins enhanced the total HBsAg expression level by about 2-fold compared with the S. cerevisiae system expressing HBsAg only, respectively (Table 3). But AHA1 and SCJ1 co-expression showed a negative effect on HBsAg production. AHA1 and SCJ1 co-expression with HBsAg expression resulted in a decrease in HBsAg concentration. And three different molecular sizes of HBsAg were found to be 25 kDa, 35 kDa, and 40 kDa by the intracellular immunoblot assay. By previous research, the protein band with 25 kDa and 35 kDa indicated the authentic HBsAg and MFα signal sequence containing HBsAg, respectively. And the protein band with 40 kDa was confirmed as N-linked glycosylated form of MFα fusion HBsAg by Endoglycosidase H (endo H) treatment. Immunoblot assay provided that HBsAg with 25 kDa was mainly expressed in S. cerevisiae 2805/MδCL-HBs. On the other hand, HBsAg bands with 40 kDa, 35 kDa, 25 kDa were simultaneously expressed in S. cerevisiae 2805/MδCL-HBs with PDI1, SEC23, TRX2 expression. That showed incomplete processing of signal peptide cleavage, which might be due to a shortage of KEX2 protease.

### Example 3

### Combinatorial Co-Expression of Protein Folding Accessories

To examine the synergistic effect of coexpresssion of folding accessory proteins on HBsAg production, 4 kinds of combinations were used among PDI1, SEC23, TRX2. For using the combinations, 3 plasmids were constructed additionally (FIG. 7).

Co-expression of the PT (PDI1+TRX2) and PS (PDI1+SEC23) combination showed total HBsAg expression levels of 31.8 and 26.3 mg/L which were 2.5 and 2.1 times higher than without co-expression, respectively (Table 4). And 3 types of HBsAg band in immunoblot assay were also observed like those of single chaperone co-expressions. In the case of the ST (SEC23+TRX2) and PST (PDI1+SEC23+TRX2) combination did not show a remarkable increase in HBsAg expression level. And a very low-level of cell growth was observed in PST combination. But PT (PDI1+TRX2) and PS (PDI1+SEC23) combinations enhanced the expression level of HBsAg.

TABLE 4
Effects of combinatorial expression of protein
folding accessories on HBsAg expression

| | Specific content of | Total HBsAg |
|---|---|---|
| Co-expression | HBsAg (mg/g cells) | content (mg/L) |
| Without co-expression | 1.62 | 12.7 |
| PDI1 + TRX2 | 3.61 | 31.8 |
| PDI1 + SEC23 | 5.22 | 26.3 |
| SEC23 + TRX2 | 2.57 | 16.1 |
| PDI1 + SEC23 + TRX2 | 3.68 | 18.2 |

### Example 4

### Investigation of Effects of KEX2-Mediated Reaction

The prosequence is subjected to being cleaved in vivo by membrane-associated KEX2 protease upon translocation into transport vesicles for cisternal packaging out of the Golgi apparatus. The co-expression of folding accessory proteins confirmed a shortage of KEX2 processing as well as enhancement of total HBsAg expression. The experiment was done to add KEX2 enzyme to combinatorial co-expression.

### 4.1. Construction of KEX2 Expression Vectors

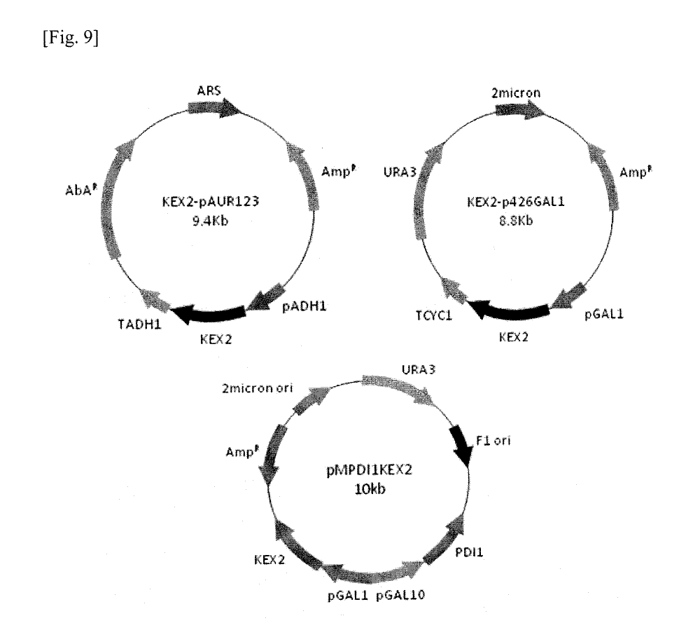

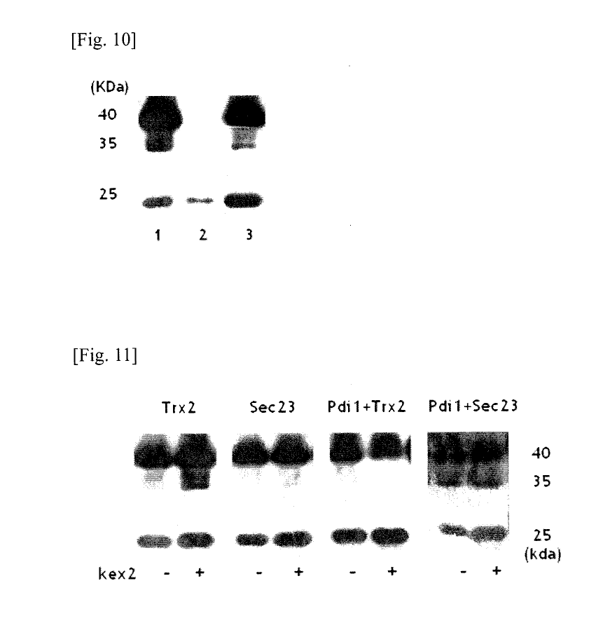

Immunoblot assay showed that MFα signal sequence fused with HBsAg gene was unprocessed. To solve this problem KEX2 was introduced. KEX2 is a serine protease able to cleave at paired dibasic sites in target peptides and is required to produce the mature forms of proteins such as alpha-factor and killer toxin. To over-express KEX2, the KEX2 gene was amplified from the S. cerevisiae 2805 chromosome and cloned into p426GAL1, pMPDI1KEX2, and pAUR123 to accommodate various selection markers and promoters for co-expression with genes of interest (FIGS. 9 and 10). The GAL1 promoter in KEX2-p426GAL1 and GAL10 promoter in pMPDI1KEX2 are inducible by galactose, and ADH1 promoter in KEX2-pAUR123 is constitutive.

### 4.2. Selection of a Suitable Promoter for Co-Expression of HBsAg

To select a suitable promoter for KEX2 expression, two recombinant strains were constructed. One is S. cerevisiae 2805/MδCL-HBs_PDI1 with constitutive KEX2 expression, and the other is S. cerevisiae 2805/MδCL-HBs_PDI1 with inducible KEX2 expression. As a result of immunoblot assay, inducible KEX2 co-expression showed significantly decrease of cell growth and HBsAg expression level (Table 5). But constitutive KEX2 co-expression increased HBsAg expression level with 25 kDa and decreased MFα containing form with 35, 40 kDa (FIG. 10). For reducing the amount of MFα containing form and increasing HBsAg expression with 25 kDa, constitutive expression promoter for KEX2 co-expression was used in the present invention.

TABLE 5
Effects of promoter for KEX2 coexpression on HBsAg expression

| | 1. control | 2. inducible | 3. constitutive |
|---|---|---|---|
| Final DCW (g/L) | 11.2 | 2.0 | 9.3 |
| 25 kDa HBsAg | 1.65 | 0.77 | 3.37 |
| concentration (mg/L) | | | |
| Total HBsAg | 11.40 | 0.77 | 11.96 |
| concentration (mg/L) | | | |

### 4.3. Effects of KEX2 Co-Expression on Expression of HBsAg

To examine the effects of KEX2 co-expression, constitutive KEX2 expression was simultaneously added to HBsAg expression with TRX2, SEC23, PT, PS combination co-expression, respectively. As a result of immunoblot assay, the protein band at 25 kDa increased about 1.4 fold by KEX2 co-expression. Like PDI1 and PDI1 with KEX2 co-expression (FIG. 10, Lane 1 and 3), other folding accessory proteins also showed increase of HBsAg expression at 25 kDa in immunoblot analysis (FIG. 11). And in the case of PDI1 and PT combination, protein bands at 35 kDa, 40 kDa was reduced. According to the immunoblot assay, KEX2 co-expression is beneficial for the further processing of the MFα signal peptide, which convert the unprocessed 35 kDa HBsAg to the processed 25 kDa HBsAg. Furthermore, it was confirmed that total HBsAg expression increased in KEX2 co-expression strains (Table 6).

TABLE 6
Effects of KEX2 coexpression on authentic HBsAg expression

| | T | TK | S | SK | PT | PTK | PS | PSK |
|---|---|---|---|---|---|---|---|---|
| 25 kDa HBsAg | 2.79 | 2.97 | 2.50 | 3.74 | 2.34 | 4.01 | 2.39 | 4.00 |
| concentration | | | | | | | | |
| (mg/L) | | | | | | | | |
| Total HBsAg | 9.49 | 10.8 | 11.1 | 10.7 | 8.57 | 7.00 | 9.42 | 11.1 |
| concentration | | | | | | | | |
| (mg/L) | | | | | | | | |

### Example 5

### Fermentation

### 5.1. Batch Fermentation

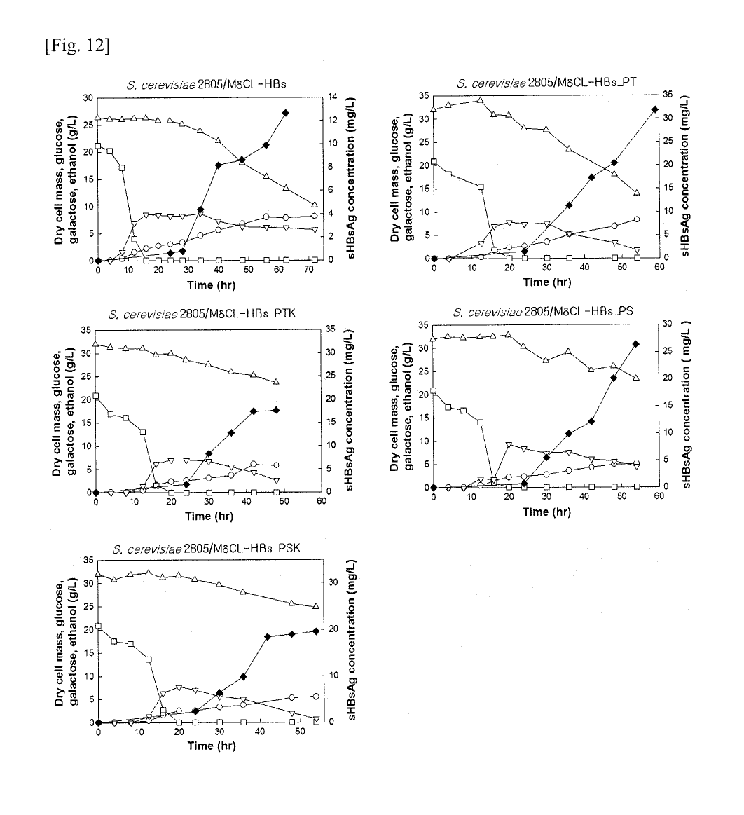

Batch fermentations were performed with five recombinant S. cerevisiae strains selected by immunoblot assay (Table 7). As a result of fermentation, the five strains did not show any discernible differences in ethanol production and glucose consumption (FIG. 12). In the case of galactose consumption, about 15 g was consumed in S. cerevisiae 2805/MδCL-HBs and S. cerevisiae 2805/MδCL-HBs_PT. But there was no significant galactose consumption in the other strains. The co-expression of genes seemed to retard cell growth except for PT combination. However, specific content of HBsAg in specific amount of cell is dramatically increased in every co-expression cases. Among them PS combination was best (Table 8).

TABLE 7
Recombinant S. cerevisiae strains used in batch fermentation

| Strains | Co-expression combinations |
|---|---|
| S. cerevisiae 2805/MδCL-HBs | None |
| S. cerevisiae 2805/MδCL-HBs_PT | PDI1, TRX2 |
| S. cerevisiae 2805/MδCL-HBs_PTK | PDI1, TRX2, KEX2 |
| S. cerevisiae 2805/MδCL-HBs_PS | PDI1, SEC23 |
| S. cerevisiae 2805/MδCL-HBs_PSK | PDI1, SEC23, KEX2 |

TABLE 8
Summary of batch fermentations results

| | Specific content of | DCW | Total HBsAg content |
|---|---|---|---|
| Co-expression | HBsAg (mg/g cell) | (g/L) | (mg/L) |
| control | 1.57 | 8.10 | 12.7 |
| PT | 3.37 | 9.45 | 31.8 |
| PTK | 3.06 | 5.76 | 17.6 |
| PS | 5.20 | 5.04 | 26.3 |
| PSK | 3.58 | 5.28 | 18.9 |

### 5.2. Optimization of Feeding Strategy for Fed-Batch Fermentation

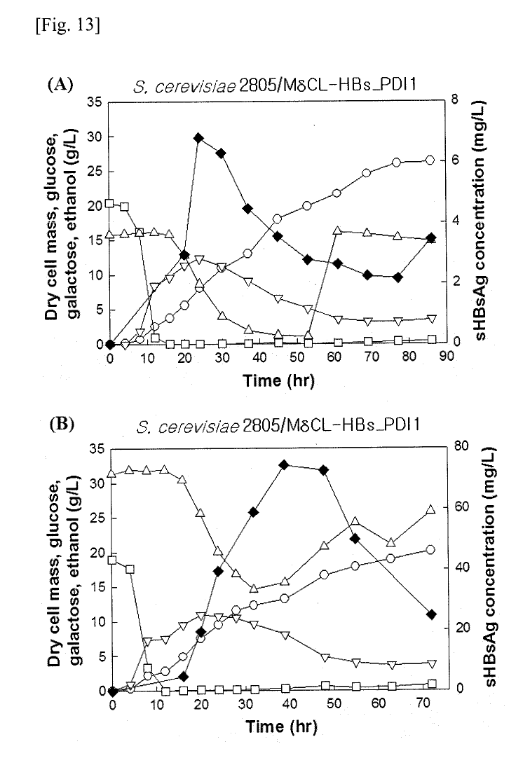

A number of fed-batch fermentations were conducted by using different feeding strategies of galactose into the growing culture in an attempt to increase HBsAg productivity. To control the galactose feeding rate in fed-batch cultures, fundamental fermentation parameters were estimated from the batch culture data (FIG. 13). Experiments were performed using the optimized medium with industrial grade chemicals and with the optimal levels of inoculum. Because the expression of HBsAg is dependent on cell growth, the cell growth had to be maximized in fed-batch fermentation. For cell growth and foreign gene expression, carbon sources such as glucose should be supplied. However, glucose, when it is abundant in the medium, causes catabolite repression (Carlson, 1999), which elicits variety of responses that ensures its preferential use, and hence, overrides induction if the GAL-mediated foreign gene is expressed. Accordingly, genetic manipulation or a discrete glucose feeding strategy is necessary to prevent glucose repression (Lee et al. 2007, J Biotechnol 126:562-567).

At these experiments, a glucose-limited fed-batch fermentation was carried out to prevent glucose repression. After the depletion of glucose, the concentrated glucose solution was fed continuously (3 ml/hr) and its concentration in the fermentor was maintained at a basal level. Because most of galactose wasn't used in the batch cultures, initial galactose concentration in medium was changed to 15 g/L and after depletion of galactose in medium, 150 g/L galactose solution was supplied intermittently (FIG. 13, A). As a result of fed-batch cultures, HBsAg expression was not induced and sharply decreased after 24 hr and galactose consumption was highly increased compared with batch cultures. After then another galactose feeding strategy was established, initial galactose concentration in the medium was changed to 30 g/L and its concentration was controlled between 15 g/L and 25 g/L, by feeding the concentrated galactose solution (250 g/L) intermittently (FIG. 13, B). The recombinant cells utilized glucose, galactose and ethanol as carbon sources and a final dry cell mass was obtained at 20 g/L.

### 5.3. Fed-Batch Fermentation

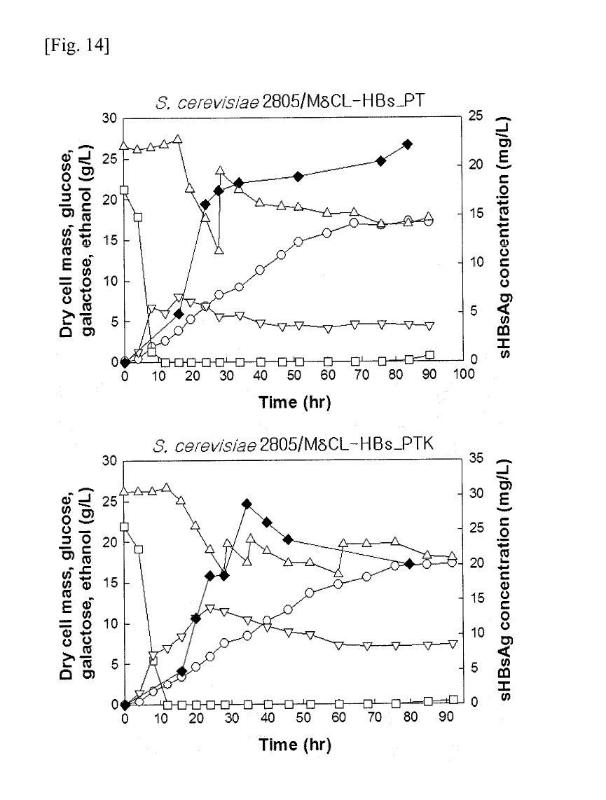

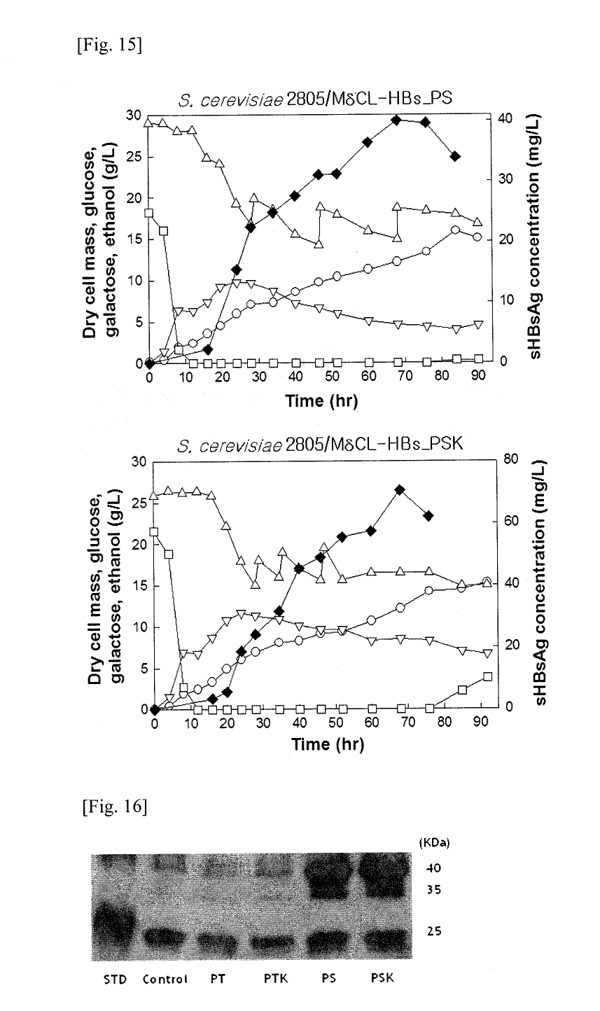

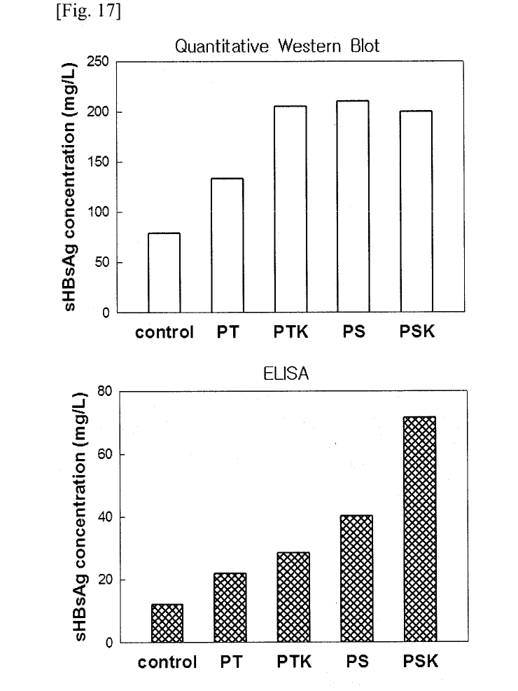

To obtain high amount of HBsAg, fed-batch fermentations of recombinant strains were carried out in YP medium initially containing 20 g/L glucose and 30 g/L galactose. After the depletion of glucose at 12 hr, the concentrated glucose solution (600 g/L) was fed continuously (3 ml/hr) and its concentration in the fermentor was maintained at a basal level. To control the galactose concentration between 15 g/L and 25 g/L, the concentrated galactose solution was supplied intermittently (FIG. 14, 16). However, there was no significant difference in cell growth, glucose consumption and ethanol production among the strains. There were three times of galactose consumption in every strains except for S. cerevisiae 2805/MδCL-HBs_PT strain. HBsAg expression increased sharply in 20 hr of culture and its maximum concentration reached 70.6 mg/L in the case of S. cerevisiae 2805/MδCL-HBs_PSK which is 12 times higher than that of no co-expression (Table 9). And to quantify the content of HBsAg containing activity as antigen, ELISA was performed. The absolute values for the amount HBsAg between the ELISA and the quantitative western blot was not well matched because of their intrinsic different assay method (FIG. 17), but it showed same trend of the enhancement of HBsAg expression level compared to the control. The three strains (co-expression with PTK, PS, PSK combination) were confirmed to have obvious binding activity against polyclonal anti-HBsAg Antibody used in the ELISA.

TABLE 9
Summary of fed-batch fermentation results

| | Specific content | | |
|---|---|---|---|
| | (mg/g cell) | Final | Total HBsAg |
| | of HBsAg | DCW (g/L) | content (mg/L) |
| control | 0.51 | 11.4 | 5.85 |
| PT | 1.29 | 17.0 | 22.2 |
| PTK | 3.41 | 17.2 | 28.8 |
| PS | 3.29 | 15.0 | 40.0 |
| PSK | 5.81 | 15.2 | 70.6 |

### Example 6

### Localization of HBsAg in S. cerevisiae

Immuno-electron microscopy has been proven to be a powerful tool to simultaneously show the precise localization of a protein and the ultra-structure of the organelle and environment wherein it is embedded (Geuze H J, 1999; Binder M et al. 1996). To analyze the localization of HBsAg in the cells, immunogold labeling was carried out by using specific antibodies and secondary antibodies tagged with colloidal gold as an electron-dense marker.

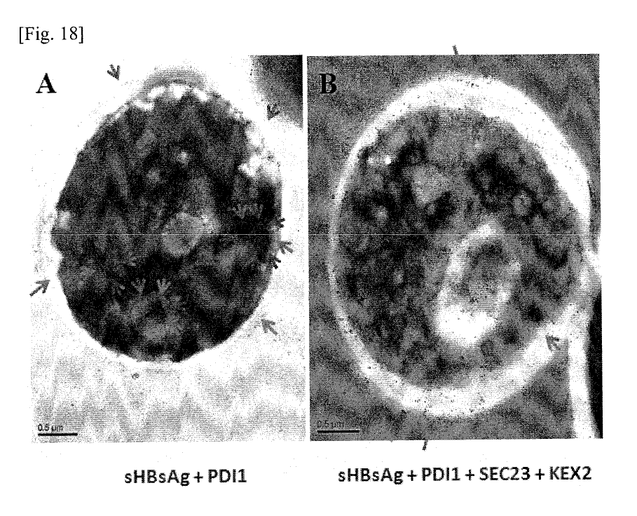

According to TEM by immune-gold labeling, it was confirmed that there was a difference in localization of HBsAg expressed. In the strains co-expressing HBsAg and PDI1, the intracellular accumulations of the nano-gold particles were observed (FIG. 18-A). On the other hand, the nano-gold particles were spread out in the cells of the PSK strains (FIG. 18-B). It is assumed the mature HBsAg could be translocated to cell membrane via the secretory pathway, and overexpressed HBsAg could be accumulated in the pathway.

While the invention has been described with respect to the above specific embodiments, it should be recognized that various modifications and changes may be made to the invention by those skilled in the art which also fall within the scope of the invention as defined by the appended claims.

1. A method for producing a target protein comprising the step of culturing a yeast transformed with: a recombinant gene construct comprising a yeast promoter, a gene coding a signal sequence, and a gene coding a target protein; and also with one or more genes coding folding accessory proteins selected from the group consisting of PDI1 (protein disulfide isomerase 1), SEC23 (secretory 23), TRX2(thioredoxin 2), AHA1 (activator of heat shock protein 90 ATPase), and SCJ1 (S. cerevisiae DnaJ); followed by the step of culturing the transformed yeast.

2. The method according to claim 1, wherein the yeast promoter is GAL 1 promoter.

3. The method according to claim 1, wherein the gene coding a signal sequence is MFα (mating factor α signal sequence).

4. The method according to claim 1, wherein the one of more genes coding folding accessory proteins are selected from the group consisting of PDI1, SEC23 and TRX2.

5. The method according to claim 1, wherein the yeast is transformed by a vector comprising both of the recombinant gene construct and the gene coding folding accessory proteins.

6. The method according to claim 1, wherein the yeast is transformed with separate vectors comprising the recombinant gene construct and one or more genes coding folding accessory proteins, respectively.

7. The method according to claim 1, wherein the yeast is further transformed with a gene coding KEX 2 (kexin 2) protease.

8. The method according to claim 1, wherein the folding accessory proteins are PDI1 and SEC23.

9. The method according to claim 1, wherein the folding accessory proteins are PDI1 and TRX2.

10. The method according to claim 1, wherein the folding accessory proteins are PDI1, SEC23, and TRX2.

11. The method according to claim 1, wherein the target protein is hepatitis B type surface antigen.

12. The method according to claim 1, wherein the yeast is Saccharomyces cerevisiae.

13. The method according to claim 1, wherein the culturing step is performed at a glucose concentration in a range of less than 1 g/L.

14. The method according to claim 1, wherein the culturing step is performed at a galactose concentration in the range of 10 g/L to 50 g/L.


SEQUENCE LISTING (16 sequences)

SEQ ID NO: 1 - DNA, 30 bp, Artificial Sequence
  Synthetic construct (F_Aha1_BamHI as primer for PCR)
cgcggatccatggtcgtgaataacccaaat

SEQ ID NO: 2 - DNA, 30 bp, Artificial Sequence
  Synthetic construct (R_Aha1_HindIII as primer for PCR)
cccaagcttttataatacggcaccaaagcc

SEQ ID NO: 3 - DNA, 34 bp, Artificial Sequence
  Synthetic construct (F_Scj1_BamHI as primer for PCR)
cgcggatccatgattccaaaattatatatacatt

SEQ ID NO: 4 - DNA, 30 bp, Artificial Sequence
  Synthetic construct (R_Scj1_HindIII as primer for PCR)
cccaagcttctacaactcatctttgagcat

SEQ ID NO: 5 - DNA, 29 bp, Artificial Sequence
  Synthetic construct (F_Sec23_BamHI as primer for PCR)
cgcggatccatggacttcgagactaatga

SEQ ID NO: 6 - DNA, 27 bp, Artificial Sequence
  Synthetic construct (R_Sec23_HindIII as primer for PCR)
cccaagcttctatgcctgaccagagac

SEQ ID NO: 7 - DNA, 30 bp, Artificial Sequence
  Synthetic construct (R_Sec23_XhoI as primer for PCR)
ccgctcgagcggctatgcctgaccagagac

SEQ ID NO: 8 - DNA, 30 bp, Artificial Sequence
  Synthetic construct (F_Trx2_BamHI as primer for PCR)
cgcggatccatggtcactcaattaaaatcc

SEQ ID NO: 9 - DNA, 29 bp, Artificial Sequence
  Synthetic construct (R_Trx2_HindIII as primer for PCR)
cccaagcttctatacgttggaagcaatag

SEQ ID NO: 10 - DNA, 32 bp, Artificial Sequence
  Synthetic construct (R_Trx2_XhoI as primer for PCR)
ccgctcgagcggctatacgttggaagcaatag

SEQ ID NO: 11 - DNA, 31 bp, Artificial Sequence
  Synthetic construct (F_Trx2_KpnI as primer for PCR)
ggggtaccccatggtcactcaattaaaatcc

SEQ ID NO: 12 - DNA, 33 bp, Artificial Sequence
  Synthetic construct (F_Kex2_ BamHI as primer for PCR)
cgggatcccgatgaaagtgaggaaatatattac

SEQ ID NO: 13 - DNA, 31 bp, Artificial Sequence
  Synthetic construct (R_Kex2_ XhoI as primer for PCR)
ccgctcgagcggtcacgatcgtccggaagat

SEQ ID NO: 14 - DNA, 35 bp, Artificial Sequence
  Synthetic construct (F_Kex2_XhoI as primer for PCR)
ccgctcgagcggatgaaagtgaggaaatatattac

SEQ ID NO: 15 - DNA, 31 bp, Artificial Sequence; identical to SEQ ID NO: 13
  Synthetic construct (R_Kex2_XhoI as primer for PCR)

SEQ ID NO: 16 - DNA, 29 bp, Artificial Sequence
  Synthetic construct (R_Kex2_XbaI as primer for PCR)
gctctagagctcacgatcgtccggaagat